(19) United States
(12) Patent Application Publication
(10) Pub. No.: US 2025/0120859 A1
(43) Pub. Date: Apr. 17, 2025

(54) ABSORBENT ARTICLES WITH IMPROVED FIT AND COMFORT

(71) Applicant: The Procter & Gamble Company, Cincinnati, OH (US)

(72) Inventors: Tanner Laurie WILLIAMS, Loveland, OH (US); Peter M. TRAN, Cincinnati, OH (US)

(21) Appl. No.: 18/904,243

(22) Filed: Oct. 2, 2024

Related U.S. Application Data
(60) Provisional application No. 63/589,655, filed on Oct. 12, 2023.

(51) Int. Cl. A61F 13/49; A61F 13/15

(52) U.S. Cl. CPC A61F 13/49012; A61F 13/49061; A61F 2013/15357

(57) ABSTRACT

An absorbent article comprising a chassis comprising a central longitudinal axis and an absorbent core is provided. The chassis comprises a front waist region, and a landing zone and an elastic waist feature both positioned in the front waist region. The elastic waist feature comprises a first end edge that is positioned proximate to a front waist edge. The elastic waist feature is: in an overlapping relationship with a portion of the landing zone by longitudinal distance, Y; and in an overlapping relationship with a portion of the absorbent core in the front waist region by a longitudinal distance, X. The elastic waist feature has a longitudinal distance, W, between the first end edge and a second end edge. A ratio of the distance, X, to the distance, W, is at least 0.4 and a ratio of the distance, Y, to the distance, W, is at least 0.4.

### CROSS REFERENCE TO RELATED APPLICATION

This application claims the benefit of U.S. Provisional Application No. 63/589,655, filed on Oct. 12, 2023, the entire disclosure of which is incorporated herein by reference.

### FIELD

The present disclosure is directed to absorbent articles with improved fit and comfort.

### BACKGROUND

Absorbent articles are worn to absorb and contain bodily exudates in infants, toddlers, children, and even adults. In order to contain the bodily exudates it is important that the absorbent articles have a good and comfortable fit around the waist and legs of a wearer. Front waist fit and leakage is one of the key problem areas to solve in absorbent articles. Further, barrier leg cuffs should be improved to provide comfort during wear while still providing an excellent barrier function.

### SUMMARY

The introduction of an elastic waist feature, such as a waistband or waist cuff, can help with front waist sagging and/or buckling by conforming more with wearer's belly, but there is room for optimization. Specifically, a longer (along on a central longitudinal axis of the absorbent article) elastic front waist feature that overlaps with other components (e.g., absorbent core, landing zone) in a front waist region can add additional stiffness to the front of the absorbent article and lessen the chance of sagging and/or buckling. One aspect of providing stiffness to the front waist region is to use an elastic waist feature with an elastic film compared to an elastic waist feature with elastic strands. The elastic film may provide greater stiffness than the elastic strands and thereby reduce front waist sagging and/or buckling. More specifically, the present disclosure provides certain overlaps between a portion of the elastic waist feature, a portion of a landing zone, and a portion of an absorbent core in the front waist region to increase the stiffness of the front waist region. The present disclosure also provides improved barrier leg cuffs that provide comfort during wear while still providing an excellent barrier function.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 9;

FIG. 12 is a plan view of an example absorbent article in the form of a taped diaper, garment-facing surface facing the viewer, in a flat laid-out state;

FIG. 13 is an example of a front waist region of a taped absorbent article with an elastic waist feature, wearer-facing surface facing the viewer;

FIG. 14 is an example of a front waist region of a taped absorbent article with reduced front waist region sagging and/or buckling, wearer-facing surface facing the viewer;

FIG. 15 is an example of a front waist region of a taped absorbent article with reduced front waist region sagging and/or buckling, wearer-facing surface facing the viewer;

FIG. 16 is an example of a front waist region of a taped absorbent article with reduced front waist region sagging and/or buckling, wearer-facing surface facing the viewer;

FIG. 17 is a cross-sectional view of FIG. 16, taken about line 17-17;

FIG. 18 is an alternative cross-sectional view of FIG. 16, taken about line 18-18;

FIG. 19 is an example cross-sectional view of an elastic waist feature, taken about line 19-19 of FIG. 17;

FIG. 20 is an example of front waist region sagging and/or buckling of comparative Example 1 when loaded with 180 mL of a saline solution;

FIG. 21 is an example of no or reduced front waist region sagging and/or buckling of the present disclosure example when loaded with 180 mL of saline solution;

FIG. 22 is an example cross-sectional view of a nonwoven material of the barrier leg cuff of FIG. 16, taken about line 22-22;

FIG. 23 is an example cross-sectional view of a nonwoven material of the barrier leg cuff of FIG. 16, taken about line 23-23;

FIG. 24A is a schematic diagram showing an example of a patterned apertured topsheet;

FIG. 24B is a schematic diagram showing a cross-section of the patterned apertured topsheet of FIG. 24A;

FIG. 24C is a schematic diagram showing an example of a patterned outer cover material; and

FIG. 24D is an example cross-sectional illustration of an absorbent article having an outer cover material and a cushion material.

### DETAILED DESCRIPTION

Various non-limiting forms of the present disclosure will now be described to provide an overall understanding of the principles of the structure, function, manufacture, and use of the absorbent articles with improved fit and comfort disclosed herein. One or more examples of these non-limiting forms are illustrated in the accompanying drawings. Those of ordinary skill in the art will understand that the absorbent articles with improved fit and comfort described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### General Description of an Absorbent Article

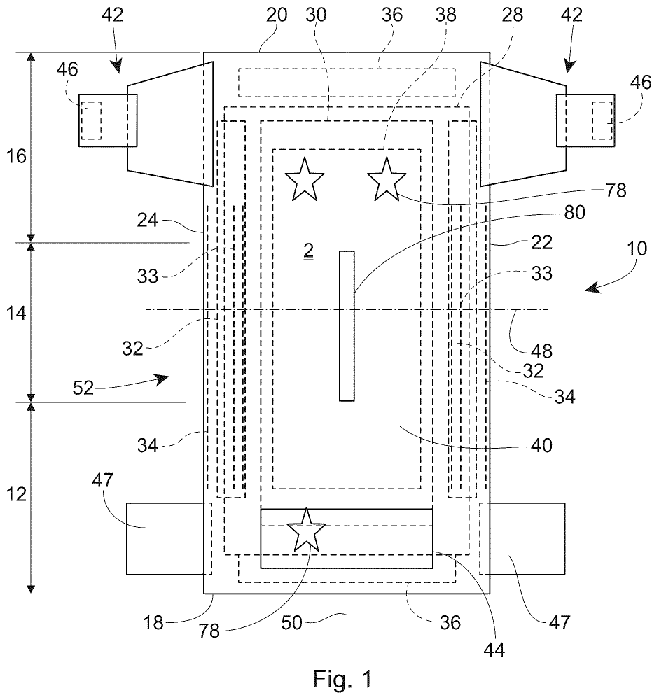

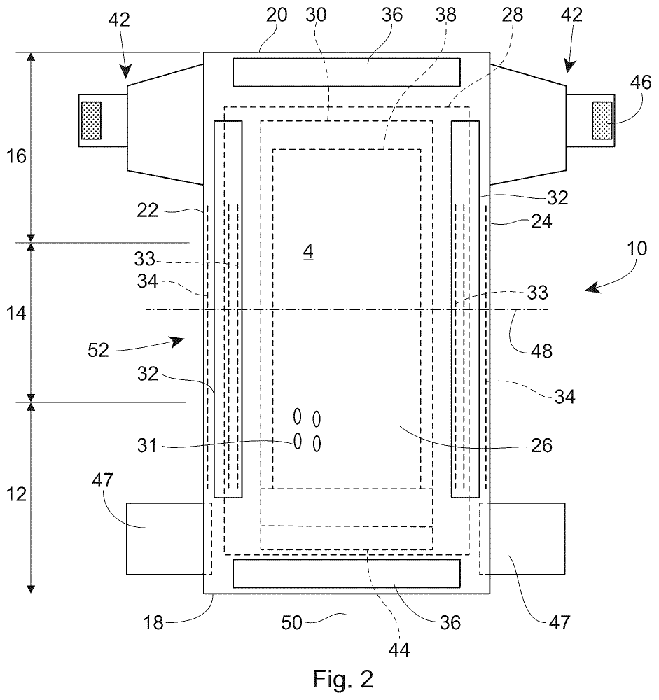

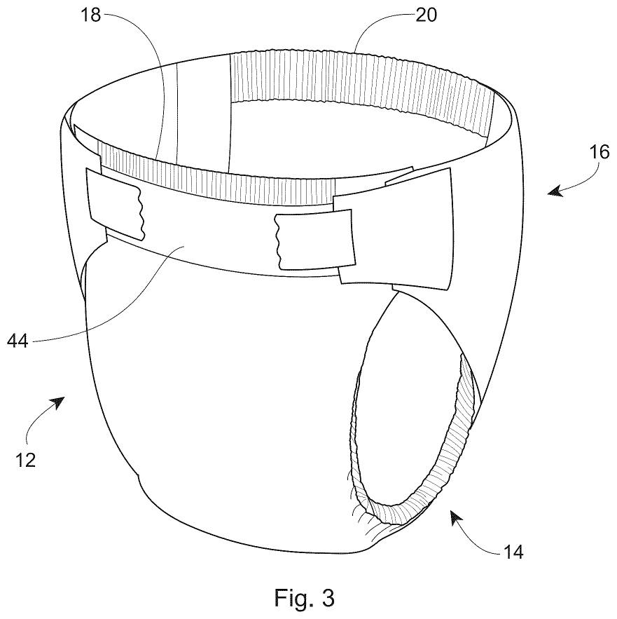

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front waist edge 18, a back waist edge 20 opposite to the front waist edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waist features, such as waistbands or waist cuffs, 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back cars 42 in the back waist region 16. The back cars 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front cars 47 in the front waist region 12. The absorbent article 10 may have a central lateral (or transverse) axis 48 and a central longitudinal axis 50. The central lateral axis 48 extends perpendicular to the central longitudinal axis 50.

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137.

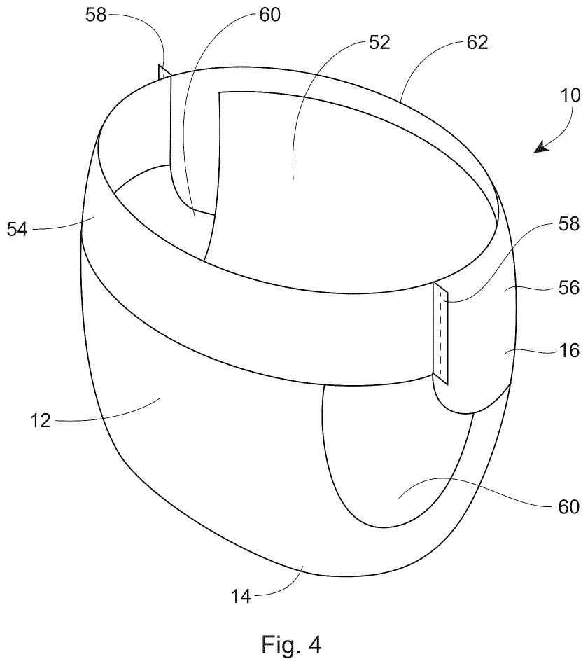

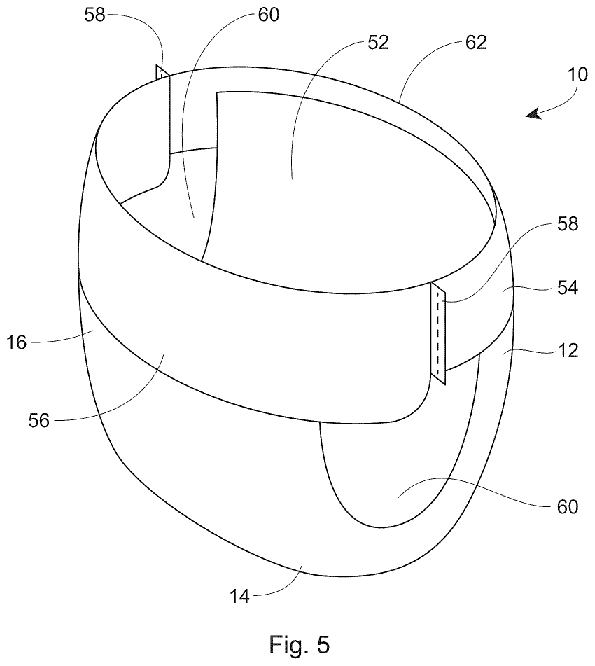

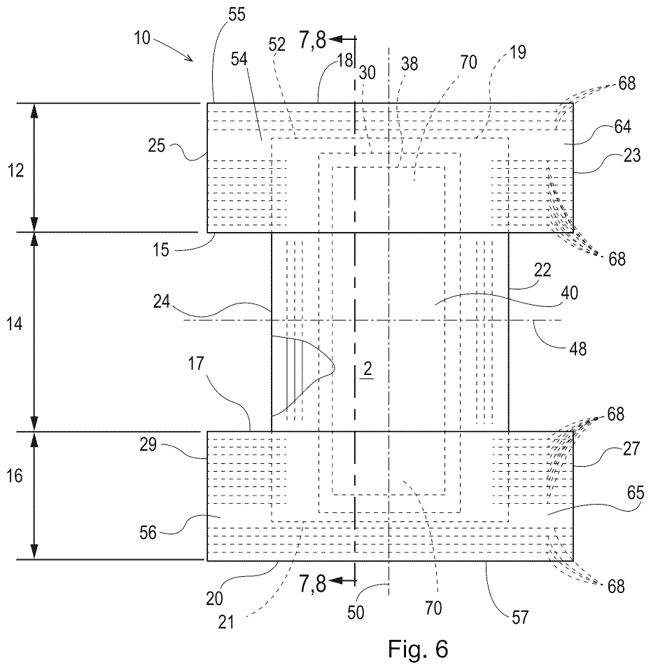

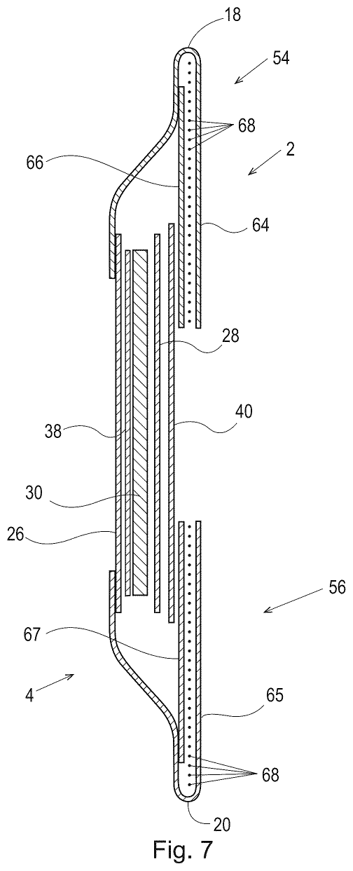

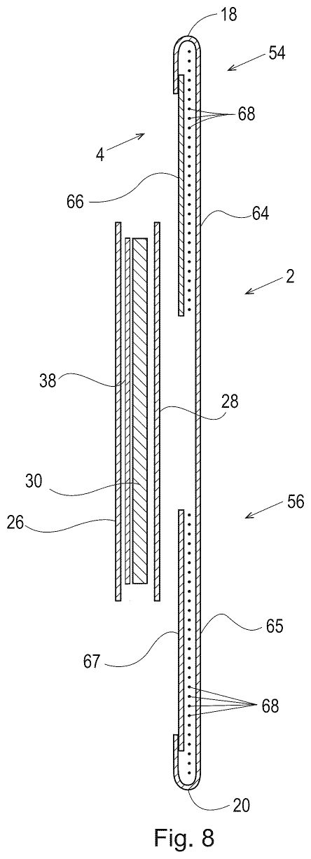

Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. No. 9,072,632.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side scams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8. In some configurations shown in FIGS. 7 and 8, portions of the outer belt layers 64, 65 may be folded over onto the inner belt layers, respectively. In addition, as shown in FIG. 7, portions of the outer belt layers 64, 65 may also be folded over onto the chassis 52.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt edges 15 and 17; or, alternatively, adjacent to the seams 58 and/or proximal front and back belt edges 15 and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may comprise a variable basis weight nonwoven material, such as those described in U.S. Pat. Appl. No. 2017/0191198. The topsheet may be apertured by overbonding a material and then rupturing the overbonds through ring rolling, such as disclosed in U.S. Pat. No. 5,628,097, to Benson et al., issued on May 13, 1997, and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet. The topsheet may comprise a one or more layer hydroentangled material with or without apertures. The topsheet may comprise a variable basis weight nonwoven material.

The topsheet may be a patterned apertured topsheet. Example topsheets with patterns of apertures are disclosed in U.S. Pat. No. 11,090,202, issued on Aug. 17, 2021, U.S. Pat. No. D851414, issued on Jun. 18, 2019, and U.S. Pat. No. D895303, issued on Sep. 8, 2020.

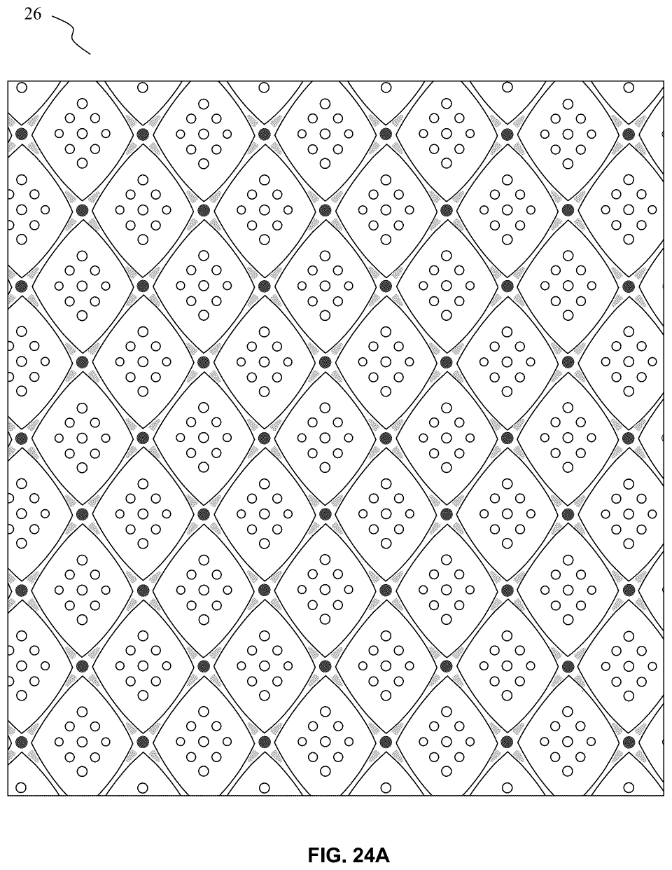

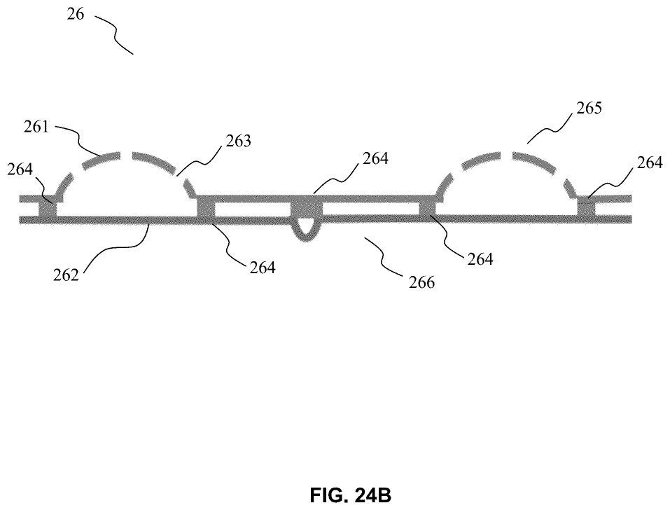

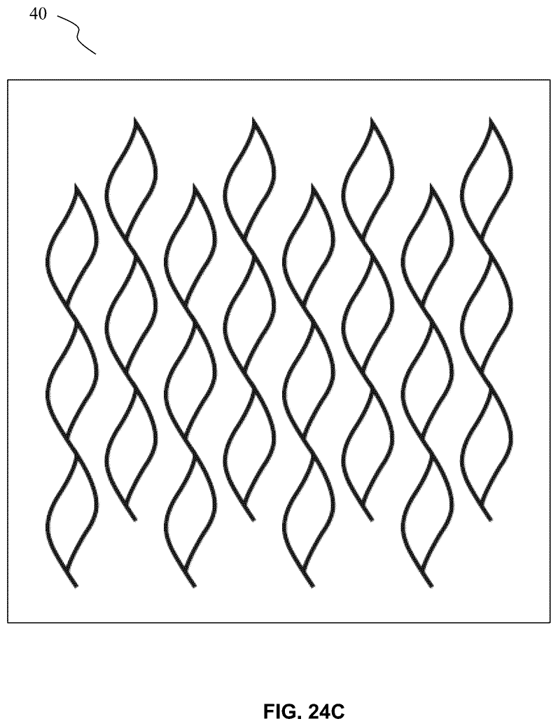

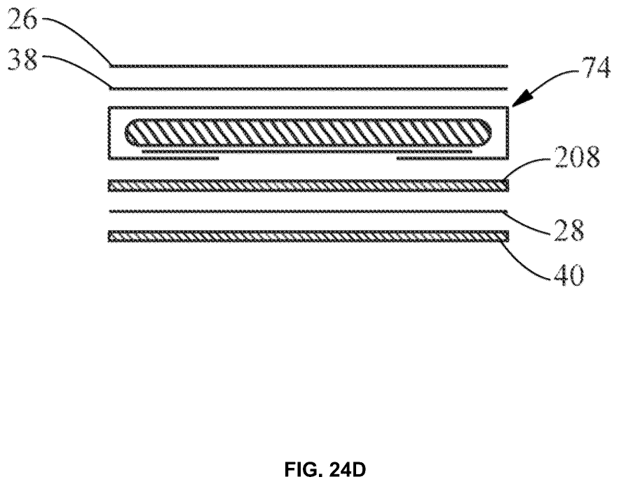

FIG. 24A is a schematic diagram showing an example of a patterned apertured topsheet.

FIG. 24B is a schematic diagram showing a cross-section of the patterned apertured topsheet of FIG. 24A. As can be seen from FIG. 24B, the topsheet 26 may comprise multiple layers, including for example a top layer 261 and bottom layer 262. The top layer 261 may be a 100% hydrophilic 2.6 denier eccentric PE/PET bicomponent fiber and may have a basis weight of 20 gsm, determined by the Basis Weight Test described hereinafter. The bottom layer 262 may be a carded nonwoven comprising 60% of 2 denier concentric hydrophilic PE/PET bicomponent fibers having a basis weight of 20 gsm and 40% of 4 denier concentric hydrophilic PE/PET bicomponent fibers having a basis weight of 42 gsm, the basis weights being determined by the Basis Weight Test described hereinafter. The bottom layer 262 may be deformed and need not be flat before the top layer is bonded to the bottom layer bonded. The topsheet 26 may include raised portions 265 and compressed regions 266. Apertures 263 in may be present in the raised portions 265 and ultrasonic bonds 264 may be present in the compressed regions 266. Other embodiments may not include ultrasonic bonds 264 in the compressed regions 266.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials, such as films, which permit vapors to escape from the absorbent article, while still preventing, or at least inhibiting, bodily exudates from passing through the backsheet. The backsheet 28 may be coterminous with the outer cover material 40.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material or a variable basis weight nonwoven material. Such a material may have one or more layers.

### Outer Cover Material Variations

The outer cover material may comprise a pattern or may have a flat, non-patterned texture. FIG. 24C is a schematic diagram showing an example of a patterned outer cover material 40. More specifically, FIG. 24C shows an outer cover material made from a carded nonwoven having an embossed pattern. The embossed pattern includes a plurality of wavy elements.

An example of a specific outer cover material that may include the pattern shown in FIG. 24C, may be a carded nonwoven comprising combination of 50% 0.8 denier PE/PET bicomponent fibers and 50% 1.2 denier PE/PET bicomponent fibers. The nonwoven may be air through cured with heat and pressure embossing, which may create the wavy pattern. An anti-static treatment may be applied to the material.

An example of a specific outer cover material that may have a flat pattern, may be a carded nonwoven comprising 50% 0.8 denier PE/PET bicomponent fibers and 50% 1.2 denier PE/PET bicomponent fibers. The fibers in the flat material may be layered in the structure. This outer cover material may be air through carded. An anti-static treatment may be applied to the material.

### Absorbent Core

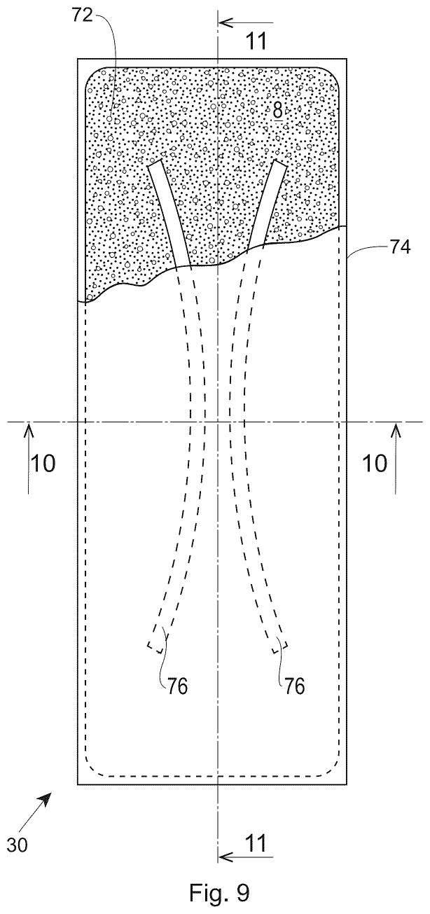

As used herein, the term “absorbent core” refers to a component of the absorbent article 10 for absorbing and containing liquid such as urine received by the absorbent article. The absorbent core thus typically has a high absorbent capacity. An example absorbent core 30 is schematically shown in FIGS. 9-11. The absorbent core comprises an absorbent material 72. The absorbent typically comprises a core wrap 74 that encloses or sandwiches the absorbent material.

The core wrap may be a single material that is folded and attached to itself, or it may comprise a separate top layer and bottom layer that may be bonded, adhesively joined, or otherwise joined together. The top and bottom layers of the core wrap may be the same or different. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be substantially free of cellulose fibers. The absorbent material may also be a high internal phase emulsion foam. High loft nonwoven materials may be present in the core bags, or proximate but outside the core bags. The absorbent material may comprise one or more layers of a coform material. The coform material may comprise a mixture of fibers and an absorbent material. The fibers may comprise staple fibers such as synthetic fibers or absorbent fibers. The synthetic fibers may comprise polypropylene fibers, polyethylene fibers, and/or bicomponent fibers. The absorbent fibers may comprise pulp, lyocell, and/or viscose. The fibers may also comprise thermoplastic filaments (scattered or interconnected networks). The absorbent material may comprise a super absorbent polymeric material in fiber or particle form. The one or more layers of coform material may comprise virgin or recycled materials. The staple fibers may be present in the coform material in an amount of from about 5 wt. % to about 50 wt. %. The absorbent material may be present in the nonwoven web in an amount of from about 50 wt. % to about 95 wt. %. The staple fibers may have an average length of from about 5 mm to about 50 mm. The staple fibers and absorbent fibers may be thermally bonded or hydraulically entangled to form the nonwoven web (as disclosed in WO2023/022979).

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from about 20 g/g to about 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, sce for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. No. 7,838,722 (Blessing), U.S. Pat. Nos. 9,072,634 and 8,206,533 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate, such as a carded nonwoven layer, having a porous structure into which SAP particles have been deposited, may also be used in present disclosure.

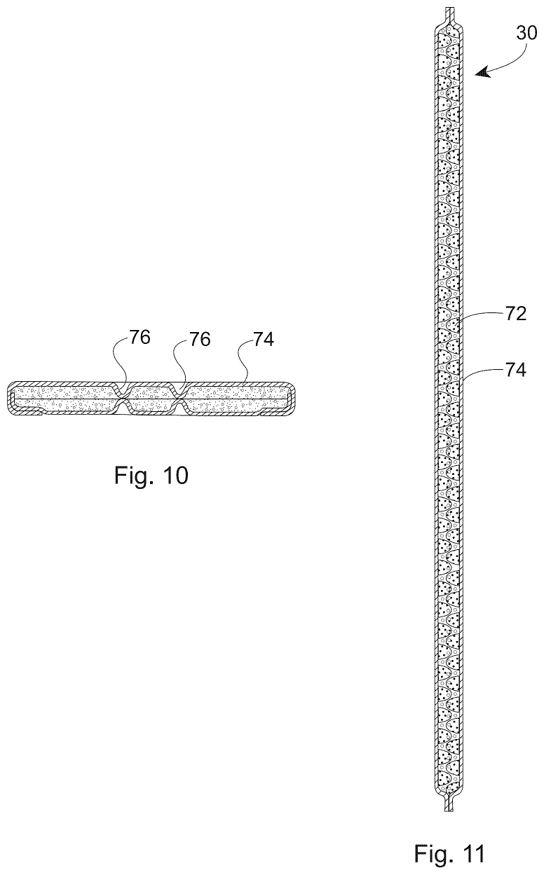

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/0312622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in U.S. Pat. No. 9,789,011. One or more channels may also be formed in absorbent cores comprising a mix of cellulose fibers and SAP particles. These channels may embody any suitable shapes and any suitable number of channels may be provided. In other instances, the absorbent core may be embossed to create the impression of channels, with absorbent material remaining within the channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front waist edge 18 and e back waist edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front waist edge 18 and the back waist edge 20. The leg elastics 34 essentially cause portions of the absorbent article 10 proximate to the chassis side edges 22, 24 to help form a seal around the legs of the wearer. The leg elastics 34 may extend at least within the crotch region 14. The barrier leg cuffs 32 may comprise one or more layers of nonwoven material. The nonwoven material may be hydroentangled.

### Elastic Waist Feature

Referring to FIGS. 1 and 2, the absorbent article 10 may comprise one or more elastic waist features 36. The one or more elastic waist features 36 may comprise a waistband or a waist cuff. The elastic waist feature 36 may be positioned on the garment-facing surface 2 or the wearer-facing surface 4. Alternatively, the elastic waist feature 35 may be positioned intermediate the topsheet and the backsheet. As an example, a first elastic waist feature 36 may be present in the front waist region 12 near the front waist edge 18 and a second elastic waist feature 36 may be present in the back waist region 16 near the back waist edge 20. The elastic waist feature 36 may aid in sealing the absorbent article 10 around a waist of a wearer and at least inhibiting bodily exudates from escaping the absorbent article 10 through the waist opening circumference. In some instances, an elastic waist feature may fully surround the waist opening circumference of an absorbent article. The elastic waist feature may comprise an elastic film joined to the topsheet and a nonwoven material covering the elastic film. In other instances, the elastic waist feature may comprise an elastic film sandwiched between two nonwoven materials. The elastic film may be ultrasonically bonded, or otherwise bonded or attached, to the one or more nonwoven materials. The one or more nonwoven materials may be hydroentangled. The clastic film and/or the nonwoven materials may be preactivated (i.e., activated prior to being joined together) or the formed elastic film/nonwoven laminate may be activated post laminate formation.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. The acquisition material may be rectangular or may be shaped, such as hourglass shaped. Typically, an acquisition material 38 may have a width and length that are smaller than the width and length of the topsheet 26. The acquisition material may be a secondary topsheet in the feminine pad context. The acquisition materials may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78. The wetness indicator may have a width in the range of about 4 mm to about 12 mm, about 6 mm to about 10 mm, or about 8 mm in a direction parallel to, or generally parallel to, the central lateral axis.

### Front and Back Ears

Referring to FIGS. 1 and 2, as referenced above, the absorbent article 10 may have front and/or back cars 47, 42 in a taped diaper context. Only one set of cars may be required in most taped diapers. The single set of cars may comprise fasteners 46 configured to engage the landing zone or landing zone area 44. If two sets of cars are provided, in most instances, only one set of the cars may have fasteners 46, with the other set being free of fasteners. Any ears with fasteners may be configured to engage an opposing car or the landing zone or landing zone area 44. One set of ears may comprise primary fasteners and the other set of ears may comprise secondary fasteners. The ears, or portions thereof, may be elastic or may have elastic panels. In an example, an elastic film or elastic strands may be positioned intermediate a first nonwoven material and a second nonwoven material. One or more of the first and second nonwoven materials may be hydroentangled. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2. The back ears may comprise an elastic film sandwiched between two nonwoven materials forming a laminate. The elastic film and/or the nonwoven materials may be pre-activated (i.e., activated prior to being joined together) and the laminate may be joined by ultrasonic bonds. Such laminates are disclosed in U.S. Pat. No. 10,485,713. In other instances, the elastic film may not be preactivated and instead the laminate may be activated after the laminate is formed. Such laminates may also be ultrasonically bonded.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Packages

The absorbent articles of the present disclosure may be placed into packages. The packages may comprise polymeric films, paper, and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages. The packages may comprise polymeric films comprising recycled material, such as about 20% to about 100%, about 30% to about 90%, about 30% to about 80%, about 40% to about 60%, or about 50% recycled material. The recycled material may comprise post-industrial recycled material (PIR) and/or post-consumer recycled material (PCR). In some instances, the polymeric films used for the packages may comprise two outer layers and one or more inner layers. The one or more inner layers may comprise the recycled material or may comprise more recycled material than the outer layers. The recycled material may comprise recycled polyethylene. The recycled material may comprise recycled polyethylene PIR from trim from the packaging operation.

The package material may comprise paper, paper based material, paper with one or more barrier layers, or a paper/film laminate. The package material may be in the range of about 50 gsm to about 100 gsm or about 70 gsm to about 90 gsm and the one or more barrier layers may be in the range of about 3 gsm to about 15 gsm. The paper based package material with or without one or more barrier layers may exhibit a machine direction tensile strength of at least 5.0 kN/m, a machine direction stretch of at least 3 percent, a cross-machine direction tensile strength of at least 3 kN/m, and a cross-direction stretch at break of at least 4 percent, each as determined via ISO 1924-3.

The paper based package material or paper based package material comprising a barrier layer or film may be recyclable or recyclable in normal paper recycling operations. The recyclability extent of the paper based package may be determined via recyclable percentage. The paper based package of the present disclosure may exhibit recyclable percentages of 70 percent or greater, 80 percent or greater, or 90 percent or greater. The paper based package of the present disclosure may have a recyclable percentage of between 70 percent to about 99.9 percent, between about 80 percent to about 99.9 percent, or between about 90 percent to about 99.9 percent. In one example, the package material of the present disclosure may exhibit a recyclable percentage of from about 95 percent to about 99.9 percent, from about 97 percent to about 99.9 percent, or from about 98 percent to about 99.9 percent. The recyclable percentage of the paper based package may be determined via test PTS-RH: 021/97 (Draft October 2019) under category II, as performed by Papiertechnische Stiftung located at Pirnaer Strasse 37, 01809 Heidenau, Germany. In another instance, the paper based packages of the present disclosure may exhibit an overall “pass” test outcome as determined by PTS-RH: 021/97 (Draft October 2019) under category II method. Any of the paper based packages may have opening features, such as lines of perforation, and may also have handles.

### Arrays

“Array” means a display of packages comprising disposable absorbent articles of different article constructions (e.g., different elastomeric materials [compositionally and/or structurally] in the side panels, back cars, side flaps and/or belts flaps, different graphic elements, different product structures, fasteners, waistbands, or lack thereof). The packages may have the same brand and/or sub-brand and/or the same trademark registration and/or having been manufactured by or for a common manufacturer and the packages may be available at a common point of sale (e.g., oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Fasteners

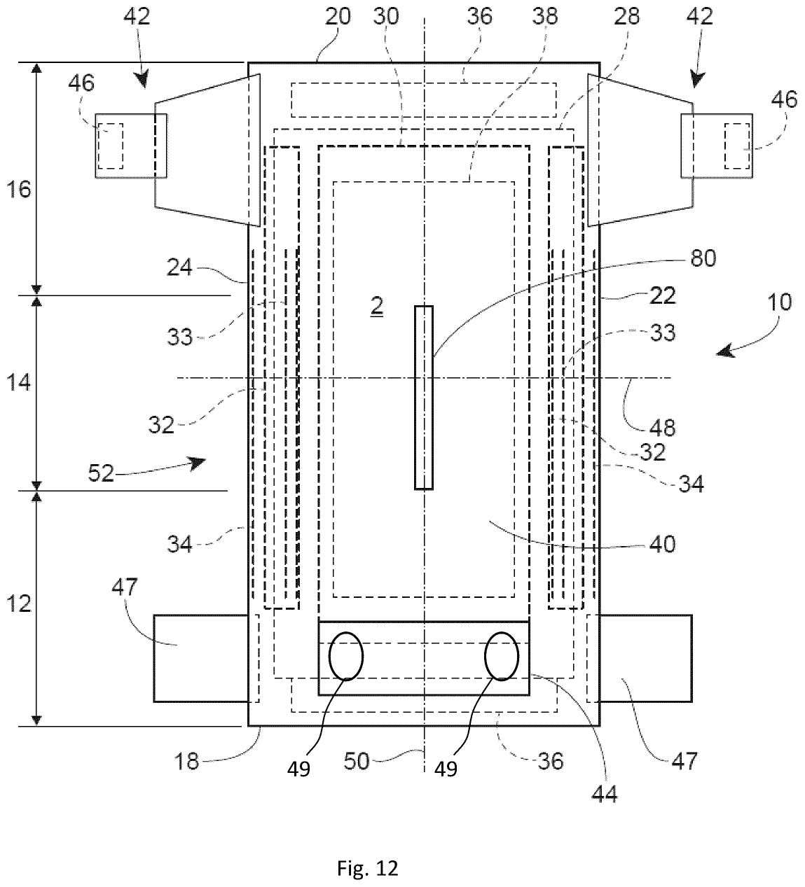

Referring to FIG. 12, the back ears 42 may comprise fasteners 46. The fasteners 46 may be configured to cooperate with the landing zone area or landing zone material 44 on the garment-facing surface of the front waist region 12 of the absorbent article 10. Additionally, the absorbent article may comprise one or more secondary fasteners 49. The secondary fasteners 49 may be disposed on the outer cover material 40 or a component of the absorbent article, such as the landing zone 44 as illustrated in FIG. 12. The secondary fasteners may be a separate material joined to the absorbent article. The secondary fasteners may be integrally formed from a material of the absorbent article. For example, the secondary fasteners may comprise one or more hooks or protrusions formed from the material of the outer cover material 40, the landing zone material 44, or a film. The hooks or protrusions may be formed using the process described in U.S. Pat. No. 8,784,722 to Rocha et al. The secondary fasteners may be configured to cooperate with a portion of the back cars. For example, a secondary fastener comprising hooks or protrusions are configured to engage the nonwoven material of the back car. It is to be appreciated that the secondary fasteners may be any mechanical fastener that is configured to cooperate with a component of the absorbent article.

### Front Waist Region Fit

Front waist fit and leakage is one of the key problem areas to solve in taped diapers. The introduction of an elasticized front waist feature can help reduce front waist gapping and buckling by conforming more with a wearer's belly, but there is much room for optimization. Specifically, a longer elastic front waist feature (longer along the central longitudinal axis 50) that overlaps with other front waist region components may add additional stiffness to the front of the diaper and lessen the chance of front waist region gapping, sagging, and/or buckling.

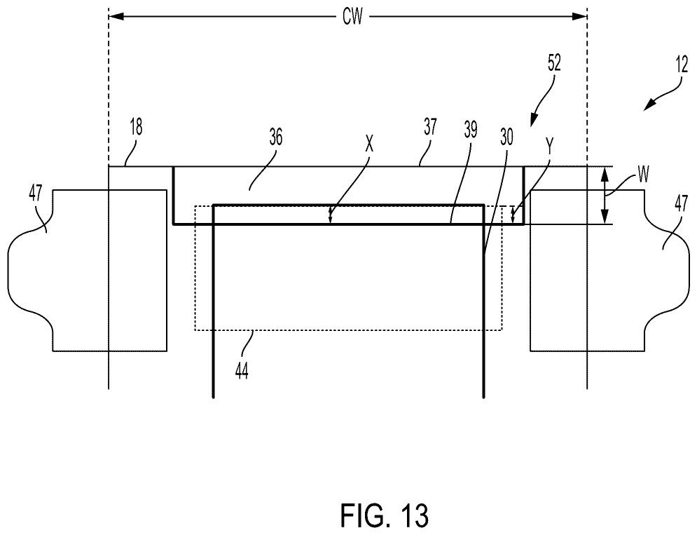

Referring to FIGS. 13-23, any reference numbers that were previously used above with reference to FIGS. 1-12 are the same as or similar to that discussed herein with respect to FIGS. 13-23.

Referring to FIG. 13, an example of a front waist region 12 of a taped absorbent article with an elastic waist feature 36 is illustrated, wearer-facing surface facing the viewer. A portion of the elastic waist feature 36 slightly overlaps (in the into the page direction) a portion of the landing zone 44 and a portion of the absorbent core 30. This slight overlap of a portion of the elastic waist feature 36, a portion of the landing zone 44, and a portion of the absorbent core 30 may lead to front waist region buckling and/or sagging when the absorbent article has been loaded with bodily exudates. The overlap of the elastic waist feature 36 with the absorbent core 30 is illustrated as distance X. The overlap of the waistband 36 with the landing zone 44 is illustrated as distance Y. A longitudinal length of the elastic waist feature 36 from a first end edge 37 to a second end edge 39 of the elastic waist feature 36 is illustrated as distance W. The slight overlap may not provide enough stiffness to the front waist region 12 to prevent or inhibit front waist region sagging and/or buckling. The present inventors have discovered that by increasing the overlap of a portion of the elastic waist feature, a portion of the absorbent core, and a portion of the landing zone, front waist region sagging and/or buckling may be avoided or reduced owing to the increased stiffness provided to the absorbent article in the front waist region 12.

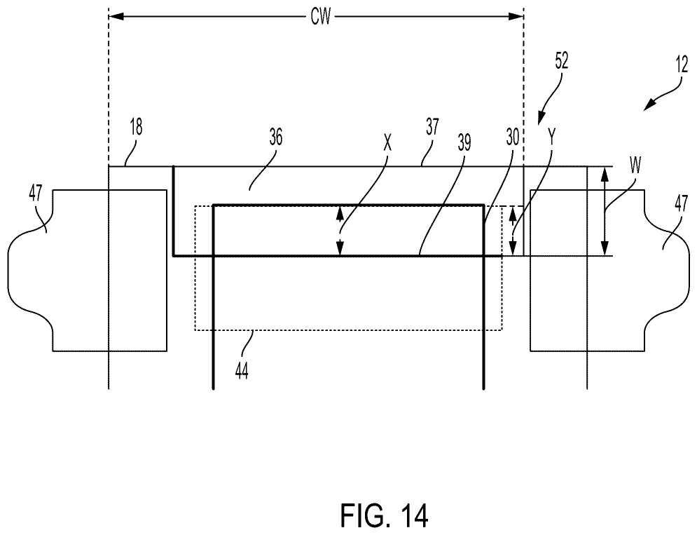

Referring to FIG. 14, an example of a front waist region 12 of a taped absorbent article with reduced front waist region 12 sagging and/or buckling is illustrated, wearer-facing surface facing the viewer. As can be seen in FIG. 14, a much greater portion of the elastic waist feature 36 (e.g., a waistband or a waist cuff) overlaps a much greater portion of the landing zone 44, and a much greater portion of the absorbent core 30 compared to FIG. 13. An overlap of the elastic waist feature 36 with the absorbent core 30 is illustrated as longitudinal distance X, in a direction parallel to the central longitudinal axis. An overlap of the elastic waist feature 36 with the landing zone 44 is illustrated as longitudinal distance Y, in a direction parallel to the central longitudinal axis. A longitudinal length of the elastic waist feature 36 from a first end edge 37 to a second end edge 39 of the elastic waist feature 36 is illustrated as total longitudinal distance W, in a direction parallel to the central longitudinal axis. A ratio of the longitudinal distance, X, to the longitudinal distance, W, is at least about 0.4, about 0.4 to about 0.95, about 0.5 to about 0.9, at least about 0.5, or about 0.5 to about 0.8. A ratio of the longitudinal distance, Y, to the longitudinal distance, W, is at least about 0.4, about 0.4 to about 0.95, about 0.5 to about 0.9, at least about 0.5, or about 0.5 to about 0.8. The longitudinal distance, W, may be about 20 mm to about 70 mm, about 30 mm to about 55 mm, or about 35 mm to about 55 mm, for example. These greater overlaps of front waist region components, provides for reduced front waist region sagging and/or buckling when the absorbent article is loaded with bodily exudates and/or during wear.

In FIGS. 13 and 14, it is noted that the front waist feature 36 is coterminous with, or substantially coterminous with (i.e., within about 0.01 mm to about 1 mm from the front waist edge 18), the front waist edge 18 of the absorbent article.

Still referring to FIG. 14, the chassis 52 may have an overall width, CW, extending in a direction parallel to the central lateral axis of the absorbent article 10. The elastic waist feature 36 may have a width extending in a direction parallel to the central lateral axis that is at least about 50% of the overall width, CW, about 50% to about 95% of the overall width, CW, or about 70% to about 90% of the overall width, CW, for example. The overall width of the chassis 52, CW, and the width of the elastic waist feature 36 are measured without elastic contraction being present. The elastic waist feature 36 may have a width extending in a direction parallel to the central lateral axis of the absorbent article 10 of at least about 100 mm, about 100 mm to about 170 mm, or about 120 mm to about 170 mm, for example. Having an elastic waist feature with a significant width extending in a direction parallel to the central lateral axis also helps stiffen the front waist region 12 of the absorbent article and helps inhibit the front waist region 12 from buckling and/or sagging during wear and/or when the absorbent article has an amount of bodily exudates present therein.

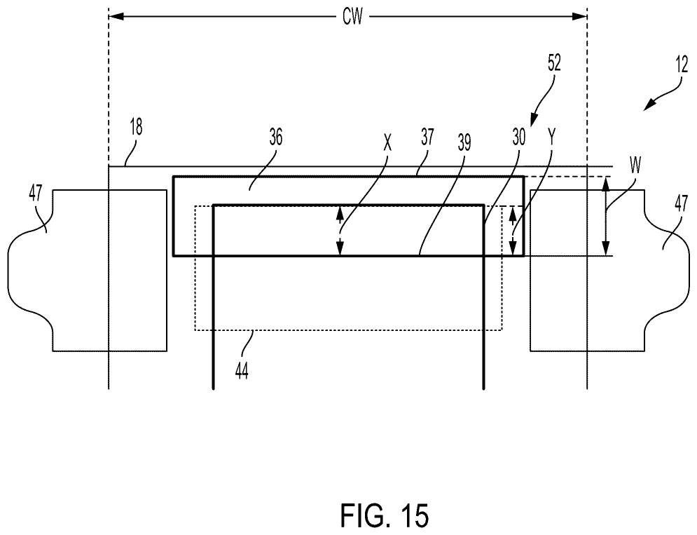

Referring to FIG. 15, the primary difference in FIG. 15 compared to FIG. 14 is that the elastic waist feature 36 is positioned proximate to the front waist edge 18 and is not coterminous or substantially coterminous with the front waist edge 18. In such an instance, distance W is measured between the first end edge 37 and the second end edge 39 of the elastic waist feature 36. The elastic waist feature 36 may typically be positioned within about 1.1 mm to about 20 mm, about 1.1 mm to about 15 mm, or about 1.1 mm to about 10 mm from the front waist edge 18.

It is important to note that the elastic waist features 36 of the present disclosure may be positioned on the wearer-facing surface 4, may be positioned on the garment-facing surface 2, or may be positioned intermediate the wearer-facing surface 4 and the garment-facing surface 2.

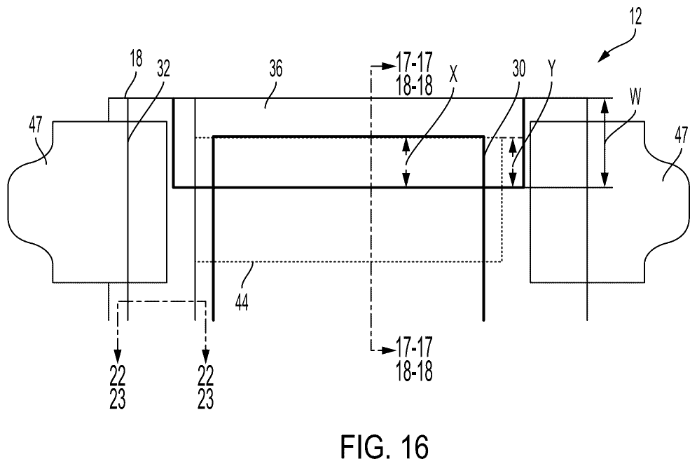

Referring to FIG. 16, the primary difference in FIG. 16 compared to FIG. 14 is that a barrier leg cuff 32 is illustrated. In FIG. 16, the wearer-facing surface is facing the viewer. Although the barrier leg cuff 32 is only illustrated on one side of the absorbent article, it will be recognized that a barrier leg cuff may also be provided on the other, opposite side. As described herein, the barrier leg cuff 32 may comprise one or more elastic strands. A portion of the barrier leg cuff 32 may overlap a portion of the elastic waist feature 36 providing additional stiffness to the front waist region 12 and reducing front waist region sagging and/or buckling.

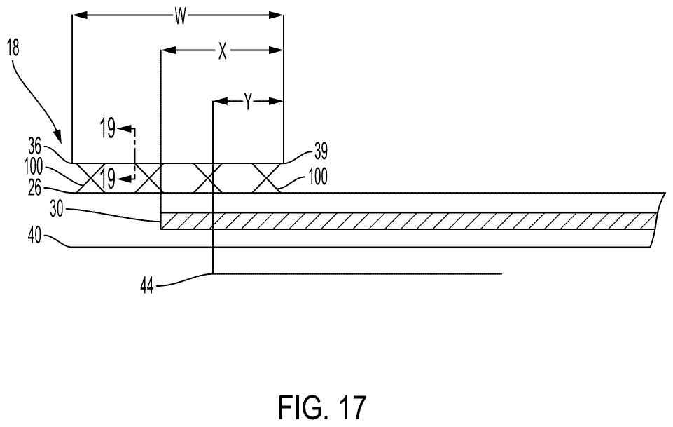

FIG. 17 is a cross-sectional view of FIG. 16, taken about line 17-17. In the form of FIG. 17, the elastic waist feature 36 comprises an elastic waistband that is joined to a wearer-facing surface of the topsheet 26. The absorbent core 30, the outer cover material 40, and landing zone 44 are also illustrated in FIG. 17. In FIG. 17, the elastic waistband 36 is bonded via bonds 100 to a wearer-facing surface of the topsheet 26. The bonds 100 are shown as X's in FIG. 17. The two middle bonds may be eliminated such that the waistband 36 is tacked down proximate to the front waist edge 18 and the second end edge 39 of the elastic waistband opposite to the front waist edge 18.

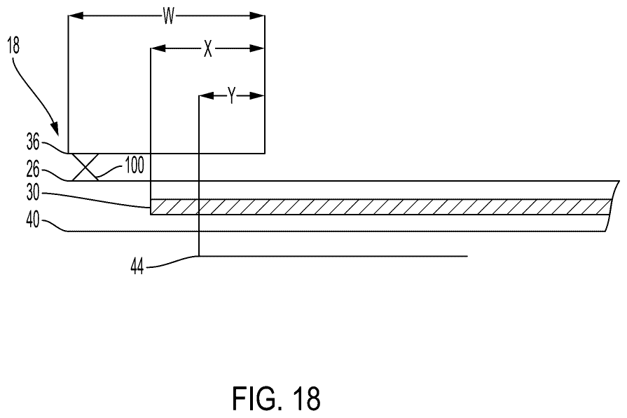

FIG. 18 is an alternative cross-sectional view of FIG. 16, taken about line 18-18. In the form of FIG. 18, the elastic waist feature 36 comprises an elastic waist cuff defining a void or pocket for receiving and containing bodily exudates. The elastic waist cuff 36 is joined to a wearer-facing surface of the topsheet 26. The absorbent core 30, the outer cover material 40, and the landing zone 44 are also illustrated in FIG. 18. In FIG. 18, the elastic waist cuff 36 is bonded via one or more bonds 100 to a wearer-facing surface of the topsheet 26. The one or more bonds 100 are shown an X's in FIG. 18. The elastic waist cuff 36 is only attached to the topsheet 26 proximate to the front waist edge and the second end edge of the elastic waist cuff is free from attachment to the topsheet 26 thereby forming a pocket with the elastic waist cuff. Having an elastic waist cuff 36 allows bodily exudates to be captured between a garment-facing surface of the elastic waist cuff 36 and a wearer-facing surface of the topsheet 26 to prevent, or at least inhibit, bodily exudates from moving up the front of the wearer past the front waist edge 18.

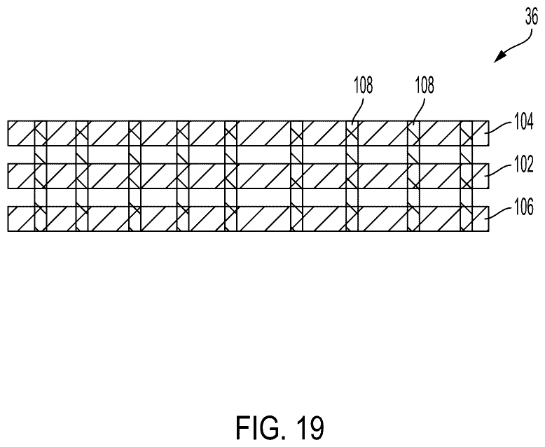

FIG. 19 is an example cross-sectional view of an elastic waist feature 36 (i.e., waistband or waist cuff) taken about line 19-19 of FIG. 17. It will be understood that the cross-sectional construction of the elastic waist feature 36 may or may not be the same if it embodies an elastic waistband or an elastic waist cuff. The elastic waist feature 36 may comprise an elastic film 102 and a first nonwoven material 104. In some instances, the elastic film 102 may be sandwiched between the first nonwoven material 104 and a second nonwoven material 106. A plurality of bonds 108 may join the first nonwoven material 104 to the second nonwoven material 106 to sandwich the elastic film 102 therebetween. Alternatively, the plurality of bonds 108 may join the first nonwoven material 104, the elastic film 102, and the second nonwoven material 106. The plurality of bonds 108 may comprise thermal bonds, mechanical bonds, chemical bonds, heat and pressure bonds, and/or ultrasonic bonds, for example. Using an elastic film 102 compared to elastic strands in an elastic waist feature 36 increases stiffness of the elastic waist feature thereby reducing sagging and/or buckling in the front waist region 12.

TABLE 1

| | | | | | | | | | Stretch | |
|---|---|---|---|---|---|---|---|---|---|---|
| | | | | | | | | Elastic | Engine in | Elastic |
| | | | | | | | | Waist | Elastic | Waist |
| Comparative | | | | | | | | Feature | Waist | Feature |
| Examples | W | X | | Y | | X/W | Y/W | Caliper | Feature | Location |
| Example 1 | 25 mm | 8 | mm | 7 | mm | 0.32 | 0.28 | 0.57 mm | Elastomeric | Attached |
| | | | | | | | | | film | to |
| | | | | | | | | | | wearer- |
| | | | | | | | | | | facing |
| | | | | | | | | | | surface |
| | | | | | | | | | | of |
| | | | | | | | | | | topsheet |
| Example 2 | 42 mm | 9 | mm | 26 | mm | 0.21 | 0.62 | 0.47 mm | Elastic | Attached |
| | | | | | | | | | strands | to |
| | | | | | | | | | | wearer- |
| | | | | | | | | | | facing |
| | | | | | | | | | | surface |
| | | | | | | | | | | of |
| | | | | | | | | | | topsheet |
| Example 3 | 23 mm | −7 | mm | −4 | mm | No | No | 0.25 mm | Extensible | Attached |
| | | | | | | overlap | overlap | | nonwoven | between |
| | | | | | | | | | | topsheet |
| | | | | | | | | | | and |
| | | | | | | | | | | backsheet |
| Example 4 | 11 mm | −5 | mm | −21 | mm | No | No | 0.70 mm | Extensible | Attached |
| | | | | | | overlap | overlap | | foam | between |
| | | | | | | | | | | topsheet |
| | | | | | | | | | | and |
| | | | | | | | | | | backsheet |
| Present | 40 mm | 23 | mm | 22 | mm | 0.58 | 0.55 | 0.57 mm | Elastomeric | Attached |

| | | |
|---|---|---|
| Disclosure | Film | to |
| Example | | wearer- |
| | | facing |
| | | surface |
| | | of |
| | | topsheet |

As can be seen in Table 1, comparative Example 1 has lower ratios of distances X/W and distances Y/W indicating lower front waist region stiffness leading to front waist region sagging and/or buckling. Comparative Example 2 indicates a lower ratio of distances X/W, a higher ratio of distances Y/W, and elastic strands indicating lower front waist region stiffness leading to front waist region sagging and/or buckling. Comparative Examples 3 and 4 indicate no overlap for distances X/W and distances Y/W indicating much lower front waist region stiffness and leading to front waist region sagging and/or buckling. The present disclosure example has both a much higher ratio of distances X/W and distances Y/W, combined with an elastomeric film, indicating much higher front waist region stiffness (due to the overlap of multiple front waist region components) and leading to much reduced front waist region sagging and/or buckling.

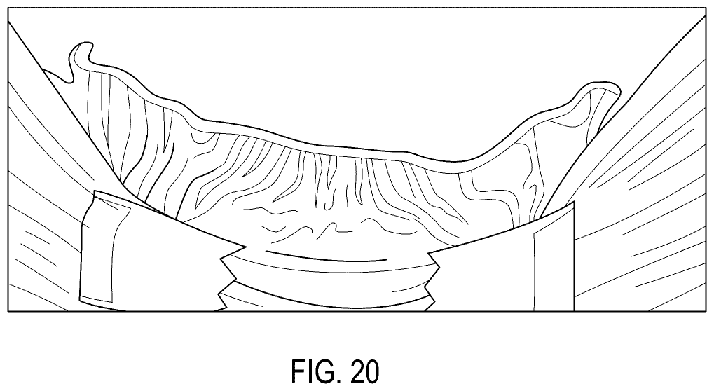

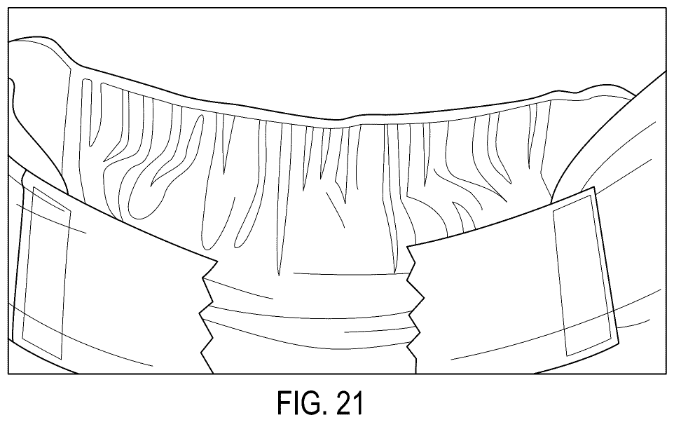

FIG. 20 is an example of front waist region sagging and/or buckling of comparative Example 1 when loaded with 180 mL of a saline solution. FIG. 21 is an example of no or reduced front waist region sagging and/or buckling of present disclosure example when loaded with 180 mL of saline solution. As can be seen, overlaps of portions of the waistband, portions of landing zone, and portions of the absorbent core of the present disclosure example significantly reduce front waist region sagging and/or buckling.

### Barrier Leg Cuff

The barrier leg cuffs of the present disclosure provided improved softness and more comfortable fit while still achieving very high Low Surface Tension Fluid Strike Through Time values because of the addition of a certain amount of meltblown fibers and Erucamide. Referring to FIG. 16, the absorbent article 10 may comprise one or more barrier leg cuffs 32 extending at least partially between the first waist edge 18 and the back waist edge 20. The barrier leg cuffs 32 may comprise one or more layers of nonwoven material and one or more elastic strands or elastic films. As an example, a barrier leg cuff may comprise a first nonwoven layer, a second nonwoven layer, a third nonwoven layer, a fourth nonwoven layer, optionally additional nonwoven layers, and one or more elastic strands. The nonwoven layers may be the same or different in fiber type, basis weight, and/or other properties. A portion of the barrier leg cuff 32 may overlap a portion of the elastic waist feature 36 to improve stiffness of the front waist region 12 and prevent or at least inhibit sagging and/or buckling of the front waist edge 18 or portions of the front waist region 12 proximate to the front waist edge 18. The barrier leg cuff 32 may have or exhibit a Low Surface Tension Fluid Strike Through Time in the range of about 8 seconds to about 35 seconds, or about 11 seconds to about 30 seconds, according to the Low Surface Tension Fluid Strike Through Time Test herein. The barrier leg cuff 32 may comprise meltblown fibers in an amount of about 12% to about 20%, or about 14% to about 18%, by weight of the nonwoven material of the barrier leg cuff. The barrier leg cuff 32 may comprise Erucamide. The Erucamide may be a melt additive and may make up about 0.5% to about 8%, about 0.5% to about 5%, or about 0.7% to about 2%, of a basis weight of the nonwoven portion of the barrier leg cuff. The nonwoven material of the barrier leg cuff may have a basis weight in the range of about 10 gsm to about 40 gsm or about 15 gsm to about 25 gsm. The barrier leg cuff 32 may comprise a plurality of bonds. The barrier leg cuff may comprise a plurality of bonds. The bonds may comprise thermal bonds, mechanical bonds, heat and pressure bonds, chemical bonds, and/or ultrasonic bonds, for example. The plurality of bonds may form a total bond area of about 10% to about 20%, about 12% to about 17%, or about 13% to about 16% relative to a total outer surface area of the barrier leg cuff.

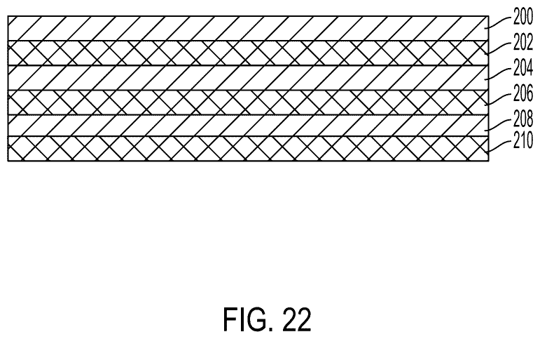

Referring to FIG. 22, an example cross-sectional view of a nonwoven material of the barrier leg cuff 32, taken about line 22-22 of FIG. 16 is illustrated. The cross-sectional view does not include clastic strands or films. The barrier leg cuff 32 may comprise, in order or not in order, a first spunbond nonwoven layer 200, a second spunbond nonwoven layer 202, a third meltblown nonwoven layer 204, a fourth meltblown nonwoven layer 206, a fifth meltblown nonwoven layer 208, and a sixth spunbond nonwoven layer 210. This can also be known as a SSMMMS nonwoven material, with S being a spunbond nonwoven layer and M being a meltblown nonwoven layer.

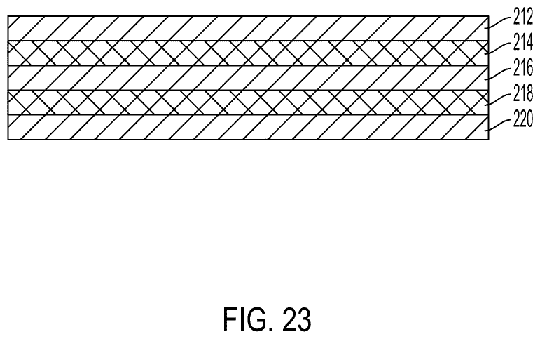

Referring to FIG. 23, an example cross-sectional view of a nonwoven material of the barrier leg cuff 32, taken about line 23-23 of FIG. 16 is illustrated. The cross-sectional view does not include elastic strands or films. The barrier leg cuff 32 may comprise, in order or not in order, a first spunbond nonwoven layer 212, a second spunbond nonwoven layer 214, a third meltblown nonwoven layer 216, a fourth meltblown nonwoven layer 218, a fifth spunbond nonwoven layer 220. This can also be known as a SSMMS nonwoven material with S being a spunbond nonwoven layer and M being a meltblown nonwoven layer.

### Cushion Material

One or more cushion materials may be provided in the absorbent articles 10 intermediate the topsheet and the backsheet. A cushion material may be a layer, laminate, or material that provides a cushiony feel when the absorbent article is touched from the garment-facing surface 2 or the wearer-facing surface 4. The cushion material may “mask” a grainy feel potentially caused by the absorbent material 72, such as superabsorbent polymers. The cushion material may also “mask” bodily exudates from being visible when viewing the wearer-facing surface 4 or the garment-facing surface 2 of the absorbent article 10. The cushion material may comprise nonwoven spunbond materials and may comprise one or more spunbond layers, such as two (SS), three (SSS), or four (SSSS), for example. The cushion material may optionally be hydroentangled. The cushion material may also not be hydroentangled, but still may comprise one more spunbond layers. The cushion material may have a basis weight in the range of about 15 gsm to about 50 gsm, about 20 gsm to about 45 gsm, about 25 gsm to about 40 gsm, about 30 to about 40 gsm, about 25 gsm, about 30 gsm, about 35 gsm, or about 40 gsm, according to the Basis Weight Test herein. The cushion material may be the layer forming the garment-facing side or the wearer-facing side of the core wrap/bag 74. The cushion material may also be placed within the core bag 74. The cushion material may be a separate material positioned intermediate the garment-facing side of the core bag 74 and the liquid impermeable backsheet 28. FIG. 24D is an example cross-sectional illustration of an absorbent article having an outer cover material and a cushion material. Referring to FIG. 24D, a cushion material 208 may be positioned intermediate a garment-facing surface of the core bag 74 and the backsheet 28. The cushion material may be free of a softening additive or may comprise a softening additive. The cushion materials, in combination with the outer cover materials discussed herein provides the absorbent articles with cushiony softness and loft. The cushion materials may have a thickness in the range of about 0.15 mm to about 0.5 mm, about 0.25 mm to about 0.30 mm, or about 0.35 mm to about 0.40 mm, or about 0.9 mm, specifically reciting all 0.1 mm increments within the specified ranges and all ranges formed therein or thereby. Hydroentangled cushion materials may be quite flexible compared to non-hydroentangled spunbond at the same basis weight. The cushion materials may have indicia printed, or otherwise applied, thereon. The cushion materials may comprise about 90% of 4 denier HDPE/PET bicomponent fibers and about 10% 3 denier PET. The cushion materials may be air through carded and may exhibit no bonding/embossing sites but only fiber-to-fiber bond intersections from the hot air bonding.

### Test Method

### Low Surface Tension Fluid Strikethrough Time Test

The low surface tension fluid strikethrough time test is used to determine the amount of time it takes a specified quantity of a low surface tension fluid, discharged at a prescribed rate, to fully penetrate a sample of a web (and other comparable barrier materials) which is placed on a reference absorbent pad. As a default, this is also called the 32 mN/m Low Surface Tension Fluid Strikethrough Test because of the surface tension of the test fluid and each test is done on two layers of the nonwoven sample simply laid on top of each other.

For this test, the reference absorbent pad is 5 plies of Ahlstrom grade 989 filter paper (10 cmx 10 cm) and the test fluid is a 32 mN/m low surface tension fluid.

### Scope

This test is designed to characterize the low surface tension fluid strikethrough performance (in seconds) of webs intended to provide a barrier to low surface tension fluids, such as runny BM, for example.

### Equipment

Lister Strikethrough Tester: The instrumentation is like described in EDANA ERT 153.0-02 section 6 with the following exception: the strike-through plate has a star-shaped orifice of 3 slots angled at 60 degrees with the narrow slots having a 10.0 mm length and a 1.2 mm slot width. This equipment is available from Lenzing Instruments (Austria) and from W. Fritz Metzger Corp (USA). The unit needs to be set up such that it does not time out after 100 seconds.

Reference Absorbent Pad: Ahlstrom Grade 989 filter paper, in 10 cmx 10 cm areas, is used. The average strikethrough time is 3.3+0.5 seconds for 5 plies of filter paper using the 32 mN/m test fluid and without the web sample. The filter paper may be purchased from Empirical Manufacturing Company, Inc. (EMC) 7616 Reinhold Drive Cincinnati, Ohio 45237.

Test Fluid: The 32 mN/m surface tension fluid is prepared with distilled water and 0.42+/−0.001 g/liter Triton-X 100. All fluids are kept at ambient conditions.
Electrode-Rinsing Liquid: 0.9% sodium chloride (CAS 7647-14-5) aqueous solution (9 g NaCl per 1 L of distilled water) is used.

### Test Procedure

Ensure that the surface tension is 32 mN/m +/−1 mN/m.
Otherwise remake the test fluid.
Prepare the 0.9% NaCl aqueous electrode rinsing liquid.
Ensure that the strikethrough target (3.3+/−0.5 seconds) for the Reference Absorbent Pad is met by testing 5 plies with the 32 mN/m test fluid as follows:
Neatly stack 5 plies of the Reference Absorbent Pad onto the base plate of the strikethrough tester.
Place the strikethrough plate over the 5 plies and ensure that the center of the plate is over the center of the paper.
Center this assembly under the dispensing funnel.
Ensure that the upper assembly of the strikethrough tester is lowered to the pre-set stop point.
Ensure that the electrodes are connected to the timer.
Turn the strikethrough tester “on” and Zero the timer.
Using the 5 mL fixed volume pipette and tip, dispense 5 mL of the 32 mN/m test fluid into the funnel.
Open the magnetic valve of the funnel (by depressing a button on the unit, for example) to discharge the 5 mL of test fluid. The initial flow of the fluid will complete the electrical circuit and start the timer. The timer will stop when the fluid has penetrated into the Reference Absorbent Pad and fallen below the level of the electrodes in the strikethrough plate.
Record the time indicated on the electronic timer.
Remove the test assembly and discard the used Reference Absorbent Pad. Rinse the electrodes with the 0.9% NaCl aqueous solution to “prime” them for the next test. Dry the depression above the electrodes and the back of the strikethrough plate, as well as wipe off the dispenser exit orifice and the bottom plate or table surface upon which the filter paper is laid.
Repeat this test procedure for a minimum of 3 replicates to ensure the strikethrough target of the Reference Absorbent Pad is met. If the target is not met, the Reference Absorbent Pad may be out of spec and should not be used.
After the Reference Absorbent Pad performance has been verified, nonwoven web samples may be tested.
Cut the required number of nonwoven web specimens. For web sampled off a roll, cut the samples into 10 cm by 10 cm sized square specimens. For web sampled off of a product, cut the samples into 15 mm by 15 mm square specimens. The fluid flows onto the nonwoven web specimen from the strike through plate. Touch the nonwoven web specimen only at the edge.
Neatly stack 5 plies of the Reference Absorbent Pad onto the base plate of the strikethrough tester. Place the nonwoven web specimen on top of the 5 plies of filter paper. Two plies of the nonwoven web specimen are used in this test method. If the nonwoven web sample is sided (i.e., has a different layer configuration based on which side is facing in a particular direction), the side facing the wearer (for an absorbent product) faces upwards in the test.
Place the strikethrough plate over the nonwoven web specimen and ensure that the center of the strikethrough plate is over the center of the nonwoven web specimen. Center this assembly under the dispensing funnel.
Ensure that the upper assembly of the strikethrough tester is lowered to the pre-set stop point.
Ensure that the electrodes are connected to the timer. Turn the strikethrough tester “on” and Zero the timer.
Run as described above.
Repeat this procedure for the required number of nonwoven web specimens. A minimum of 5 specimens of each different nonwoven web sample is required. The average value is the 32 mN/m low surface tension strikethrough time in seconds.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. An absorbent article comprising:
a chassis comprising:
a central lateral axis;
a central longitudinal axis;
a liquid permeable topsheet;
a liquid impermeable backsheet;
an absorbent core positioned intermediate the topsheet and the backsheet, wherein the absorbent core comprises an absorbent material;
a front waist region comprising a front waist edge;
a back waist region comprising a back waist edge;
a garment-facing surface;
a wearer-facing surface;
a landing zone positioned on the garment-facing surface in the front waist region; and
an elastic waist feature in the front waist region, the elastic waist feature comprising an elastic film, a nonwoven material, and a plurality of bonds joining the elastic film and the nonwoven material;
the elastic waist feature comprising a first end edge that is positioned proximate to the front waist edge, preferably, that is substantially coterminous with the front waist edge;
the elastic waist feature comprising a second end edge, the elastic waist feature is:
in an overlapping relationship with a portion of the landing zone by longitudinal distance, Y, in a direction parallel to the central longitudinal axis; and
in an overlapping relationship with a portion of the absorbent core in the front waist region by a longitudinal distance, X, in a direction parallel to the central longitudinal axis;
the elastic waist feature having a total longitudinal distance, W, between the first end edge and the second end edge, in a direction parallel to the central longitudinal axis;
wherein a ratio of the longitudinal distance, X, to the longitudinal distance, W, is at least 0.4; and
wherein a ratio of the longitudinal distance, Y, to the longitudinal distance, W, is at least 0.4.

2. The absorbent article of claim 1, wherein the ratio of the longitudinal distance, X, to the longitudinal distance, W, is at least 0.5 and less than 0.9.

3. The absorbent article of claim 1, wherein the ratio of the longitudinal distance, Y, to the longitudinal distance, W, is at least 0.5 and less than 0.9.

4. The absorbent article of claim 1, wherein the longitudinal distance, W, is about 20 mm to about 70 mm, preferably about 30 mm to about 55 mm, and more preferably about 35 mm to about 55 mm.

5. The absorbent article of claim 1, wherein the plurality of bonds comprise ultrasonic bonds.

6. The absorbent article of claim 1, wherein the second end edge has a portion free of attachment to the topsheet.

7. The absorbent article of claim 1, wherein the second end edge is joined to the topsheet throughout its length.

8. The absorbent article of claim 1, wherein the elastic waist feature has a width extending in a direction parallel to the central lateral axis of at least about 100 mm, preferably about 120 mm to about 170 mm.

9. The absorbent article of claim 1, wherein the chassis has an overall width extending in a direction parallel to the central lateral axis, and wherein the elastic waist feature has a width extending in a direction parallel to the central lateral axis that is at least about 50% of the overall width, preferably about 70% to about 90% of the overall width.

10. The absorbent article of claim 1, comprising a barrier leg cuff joined to the absorbent article, wherein the barrier leg cuff comprises a first nonwoven layer, a second nonwoven layer, a third nonwoven layer, a fourth nonwoven layer, and one or more elastic strands.

11. The absorbent article of claim 10, wherein a portion of the barrier leg cuff overlaps a portion of the elastic waist feature to impart stiffness to the front waist region.

12. The absorbent article of claim 10, wherein the barrier leg cuff exhibits a Low Surface Tension Strike Fluid Through Time in the range of about 8 seconds to about 35 seconds, preferably about 11 seconds to about 30 seconds, according to the Low Surface Tension Fluid Strike Through Time Test.

13. The absorbent article of claim 10, wherein barrier leg cuff comprises meltblown fibers in an amount of about 12% to about 20%, preferably about 14% to about 18% by weight of a nonwoven material of the barrier leg cuff.

14. The absorbent article of claim 10, wherein the barrier leg cuff comprises Erucamide, and wherein nonwoven layers of the barrier leg cuff have a total basis weight in the range of about 15 gsm to about 25 gsm.

15. The absorbent article of claim 10, wherein the barrier leg cuff comprises a plurality of bonds, and wherein the plurality of bonds form a total bond area of about 12% to about 17%, preferably about 13% to about 16% of a total area relative to a total area of the barrier leg cuff.

16. The absorbent article of claim 1, comprising a barrier leg cuff comprising, in order, a first spunbond nonwoven layer, a second spunbond nonwoven layer, a third meltblown nonwoven layer, a fourth meltblown nonwoven layer, a fifth meltblown nonwoven layer, and a sixth spunbond nonwoven layer.

17. The absorbent article of claim 1, comprising a barrier leg cuff comprising, in order, a first spunbond nonwoven layer, a second spunbond nonwoven layer, a third meltblown nonwoven layer, a fourth meltblown nonwoven layer, a fifth spunbond nonwoven layer.

18. The absorbent article of claim 1, wherein the absorbent core is substantially free of cellulose fibers, preferably, free of cellulose fibers.
